(19) United States
(12) Patent Application Publication
(10) Pub. No.: US 2024/0058252 A1
(43) Pub. Date: Feb. 22, 2024

(54) COSMETIC METHOD AND COMPOSITION BASED ON SHORT-CHAIN FRUCTOOLIGOSACCHARIDES AND A NATIVE STARCH

(71) Applicants: BEGHIN MEIJI, MOUSSY-LE-VIEUX (FR); TEREOS STARCH & SWEETENERS EUROPE, MOUSSY-LE-VIEUX (FR)

(72) Inventors: Cindy LE BOURGOT, MOUSSY-LE-VIEUX (FR); Nicolas METZGER, MOUSSY-LE-VIEUX (FR); Claire MEUNIER, MOUSSY-LE-VIEUX (FR); Perrine SY, MOUSSY-LE-VIEUX (FR)

(73) Assignees: BEGHIN MEIJI, MOUSSY-LE-VIEUX (FR); TEREOS STARCH & SWEETENERS EUROPE, MOUSSY-LE-VIEUX (FR)

(21) Appl. No.: 18/260,179

(22) PCT Filed: Jan. 12, 2022
(86) PCT No.: PCT/EP2022/050566

§ 371 Date: Jun. 30, 2023

(30) Foreign Application Priority Data
Jan. 12, 2021 (FR) 2100251
Nov. 18, 2021 (FR) 2112189

(51) Int. Cl. A61K 8/73; A61Q 17/00; A61Q 17/04; A61Q 5/02; A61K 8/60

(52) U.S. Cl. CPC A61K 8/732; A61Q 17/005; A61Q 17/04; A61Q 5/02; A61K 8/60

(57) ABSTRACT

A cosmetic method for balancing the skin microbiota. The cosmetic method includes the application to all or part of the skin, skin appendages and/or mucous membranes of at least one short-chain fructo-oligosaccharide and at least one native starch. Also, a cosmetic composition, which includes, in a cosmetically acceptable carrier, at least one short-chain fructo-oligosaccharide and at least one native starch.

### FIELD

The present invention is in the field of cosmetics. More specifically, the present invention is in the field of maintaining the balance of the skin microbiota.

### BACKGROUND

The skin is a complex and dynamic ecosystem representing the largest organ of the human body. In addition to the physical barrier, the skin microbiota guarantees protection and a biological barrier by competing with pathogens and communicating closely with the cells and components of the immune system. Thus, the cutaneous microbiota can be considered as an essential actor for the maintenance of healthy skin (1). The skin microbiota includes resident microorganisms that are commonly found in the skin that are considered commensal, meaning they are generally harmless and most likely provide some benefit to the host (2, 3). Among the resident species of skin microbiota are propionic bacteria like Propionibacterium acnes, also called Cutibacterium acnes, coagulase negative staphylococci like Staphylococcus epidermidis and other types like Corynebacteria, Micrococci and Acinebacter. S. epidermidis is a commensal bacterium, part of the skin microbiota that can be described as “normal”, which serves to protect human skin from infections and other environmental aggressions (4). C. acnes has been linked to the skin condition of acne and associated with many skin conditions (5, 6). The skin microbiota also includes transient microorganisms, the most common species of which are Staphylococcus aureus, Escherichia coli and Pseudomonas aeruginosa (4, 7). S. aureus has been identified as an important pathogen in skin overcolonization and is implicated in the development of human skin diseases (2). The composition and abundance of skin microbiota varies widely across body parts, between individuals, and over time, resulting in an extremely dynamic and highly fluctuating bacterial community (8, 9).

The human skin microbiota has recently become a center of interest for the dermatological and cosmetic fields. Understanding the skin microbiota and how to maintain its delicate balance are essential steps in understanding the mechanisms responsible for healthy skin and its appearance. Imbalances in the composition of the skin microbiota, also called dysbiosis, are associated with several skin pathologies such as acne, eczema or allergies, as well as non-pathological conditions such as sensitive skin, irritation or dry skin.

Consequently, the development of approaches preserving or restoring the balance of the microbiota represents a new target for promoting skin health. There is therefore a need for a simple and effective method to balance the microbiota of the skin.

In this context, the Applicant has demonstrated that the cosmetic use of a cosmetic composition comprising at least one short-chain fructo-oligosaccharide makes it possible to balance the cutaneous microbiota selectively, by stimulating the beneficial organisms and by limiting non-beneficial organisms. This cosmetic use makes it possible to improve the appearance and/or the comfort of the skin, appendages and/or mucous membranes.

### SUMMARY

Thus, the present invention relates to a cosmetic method for balancing the skin microbiota, comprising a step of applying to all or part of the skin, skin appendages and/or mucous membranes a cosmetic composition comprising, in a cosmetically acceptable support, at least one short-chain fructo-oligosaccharide and at least one native starch.

In one embodiment, the cosmetic method according to the invention is for improving the skin appearance and/or skin comfort.

In one embodiment of the invention, improving the appearance and/or skin comfort comprises reducing at least one skin discomfort chosen from the group consisting of redness, tightness, dull skin and itching.

In one embodiment, the at least one fructo-oligosaccharide is derived from sugar beet.

In one embodiment, the at least one short-chain fructo-oligosaccharide is a mixture:
a fructo-oligosaccharide containing one glucose unit and two fructose units, also called 1-kestose or GF2, with a degree of polymerization equal to 3;
a fructo-oligosaccharide containing one glucose unit and three fructose units, also called nystose or GF3, with a degree of polymerization equal to 4; And
a fructo-oligosaccharide containing one glucose unit and four fructose units, also called 1-beta-fructofuranosylnystose or GF4, with a degree of polymerization equal to 5.

In one embodiment, the at least one native starch is at least one native corn starch.

In one embodiment, the at least one native starch is included in the cosmetic composition in an amount ranging from 0.5 to 20% by weight relative to the total weight of the cosmetic composition and/or the at least one short-chain fructo-oligosaccharide is included in the cosmetic composition in an amount of at least 0.5% by weight relative to the total weight of the cosmetic composition.

In one embodiment of the invention, balancing the skin microbiota comprises promoting the development and/or the growth of Staphylococcus epidermidis, and/or limiting the development and/or growth of at least one organism among Cutibacterium acnes and Staphylococcus aureus.

In one embodiment, the cosmetic method according to the invention is implemented after a dysbiotic event.

The present invention also relates to the use of a cosmetic composition comprising, in a cosmetically acceptable carrier, at least one short-chain fructo-oligosaccharide, and at least one native starch, to balance the skin microbiota.

The present invention also relates to a cosmetic composition comprising, in a cosmetically acceptable carrier, at least one short-chain fructo-oligosaccharide and at least one native starch, in which the at least one short-chain fructo-oligosaccharide is a mixture:
a fructo-oligosaccharide containing one glucose unit and two fructose units, also called 1-kestose or GF2, with a degree of polymerization equal to 3;
a fructo-oligosaccharide containing one glucose unit and three fructose units, also called nystose or GF3, with a degree of polymerization equal to 4; and
a fructo-oligosaccharide containing one glucose unit and four fructose units, also called 1-beta-fructofuranosylnystose or GF4, with a degree of polymerization equal to 5.

In one embodiment, the at least one native starch is a native corn starch.

According to one embodiment, the at least one native starch is included in an amount ranging from 0.5 to 20% by weight or more than 5% to 20% by weight relative to the total weight of the cosmetic composition. According to one embodiment, the at least one short-chain fructo-oligosaccharide is included in an amount of at least 0.5% by weight relative to the total weight of the cosmetic composition.

In one embodiment, the cosmetic composition is in the form of a dermo-cosmetic product, a hair product, an intimate hygiene product, a dressing and/or a healing product.

### BRIEF DESCRIPTION OF THE DRAWINGS

FIG. 1 is a graph illustrating the growth of S. epidermidis in the presence of various amounts of scFOS at 0 h, 8 h and 24 h.

FIG. 2 is a graph illustrating the growth of C. acnes in the presence of various amounts of scFOS at 0 h, 8 h and 24 h.

FIG. 3 is a graph illustrating the growth of S. aureus in the presence of various amounts of scFOS at 0 h, 8 h and 24 h.

FIG. 4 is a graph illustrating the growth of S. epidermidis in the presence of various amounts of scFOS when it competes with C. acnes at 0 h, 8 h and 24 h.

FIG. 5 is a graph illustrating the growth of C. acnes in the presence of various amounts of scFOS when it competes with S. epidermidis at 0 h, 8 h and 24 h.

FIG. 6 is a graph illustrating the growth of S. epidermidis in the presence of various amounts of scFOS when competing with S. aureus at 0 h, 8 h and 24 h.

FIG. 7 is a graph illustrating the growth of S. aureus in the presence of various amounts of scFOS when competing with S. epidermidis at 0 h, 8 h and 24 h.

FIG. 8 is a graph illustrating the force (in Pa) to be applied to break the cream, which reflects its viscosity, and the deformation (in %) of the moisturizing creams according to the invention. The histograms correspond to the deformation, while the curve corresponds to the viscous force.

FIG. 9 is a graph illustrating the viscosity in mPa·s of the moisturizing creams according to the invention during their spreading at 100 s−1 at 25° C.

FIG. 10 is a histogram illustrating the firmness and the stickiness, in grams, of the moisturizing creams according to the invention.

FIG. 11 is a graph illustrating the force (in Pa) to be applied to break the cream, which reflects its viscosity, and the deformation (in %) of the sun creams according to the invention. The histograms correspond to the deformation, while the curve corresponds to the viscous force.

FIG. 12 is a graph illustrating the viscosity in mPa·s of the sunscreens according to the invention during their spreading at 100 s−1 at 25° C.

FIG. 13 is a histogram showing the humidity values (in %) of the solid shampoos according to the invention after 48 h at 105° C., for different amounts of short-chain fructo-oligosaccharides. The reference (Ref.) does not include short chain fructo-oligosaccharides.

FIG. 14 is a histogram showing the results of the test for dissolving solid shampoos according to the invention after 10 minutes of stirring at 250 revolutions per minute at 38° C., for different amounts of short-chain fructo-oligosaccharides. The reference (Ref.) does not include short chain fructo-oligosaccharides.

### DETAILED DESCRIPTION

In the present invention, the following terms have the following meanings:

By “improving the appearance and/or comfort of the skin, skin appendages and/or mucous membranes”, is meant reducing, or even eliminating, the number, extent, occurrence and/or intensity of at least one inconvenience of the skin, the skin appendages and/or the mucous membranes, referred to as cutaneous inconvenience. The skin discomfort can in particular be chosen from the group consisting of redness, tightness, dry skin, sensitive skin, itching, irritation and dull skin.

The term “starch” denotes a polysaccharide composed of chains of D-glucose molecules, the glucose units being linked at approximately 95% with alpha-1,4 bonds and at approximately 5% with alpha-1,6-glucose bonds. The two constituent homopolymers of starch are amylose (which results from the linear chaining of glucose units linked by alpha-1,4 bonds) and amylopectin (which has a branched structure in which linear fragments of amylose type are linked by alpha-1,6 bonds); due to the presence of amylopectin, the starch is partially branched.

The term “native starch” denotes a starch which has not undergone any modification after extraction from the plant.

The term “between X and Y” denotes the range of values between X and Y, the terminals X and Y being included in said range.

By “approximately” a nominal value, is meant in the present invention an interval between plus and minus 10% of the nominal value, preferably between plus and minus 5% of the nominal value, in particular between plus and minus 1% of the nominal value.

By “balancing the skin microbiota”, is meant the action of maintaining, preserving and/or restoring the balance of the skin microbiota. Balancing the skin microbiota includes promoting the development and/or growth of at least one organism considered beneficial to the skin, to the skin appendages and/or the mucous membranes, and/or limiting the development and/or growth of at least one organism considered not beneficial to the skin, the skin appendages and/or the mucous membranes, and/or restoring the diversity of organisms on the surface of the skin, the skin appendages and/or the mucous membranes. In some embodiments, balancing the skin microbiota includes promoting the development and/or growth of Staphylococcus epidermidis, and/or limiting the development and/or growth of at least one of Cutibacterium acnes and Staphylococcus aureus. In a particular embodiment, balancing the skin microbiota includes promoting the development and/or growth of Staphylococcus epidermidis, and limiting the development and/or growth of Cutibacterium acnes and Staphylococcus aureus.

The term “dysbiotic event” or “dysbiosis” denotes an event during which the balance of the skin microbiota is altered, either by growth and/or development of at least one organism considered not beneficial to the skin, to the skin appendages and/or the mucous membranes, or by reduction and/or disappearance of at least one organism considered beneficial to the skin, the skin appendages and/or the mucous membranes. Dysbiosis can be the cause of many inconveniences and skin pathologies. In the event of dysbiosis, the immune responses of the skin, skin appendages and/or the mucous membranes may be modified and certain pathogenic bacterial species may develop further.

Among the dysbiotic events, mention may be made in particular of those resulting from skin pathologies such as acne, psoriasis, hidradenitis suppurativa or Verneuil's disease and atopic dermatitis. In some embodiments, the dysbiotic event is acne and the microbiota imbalance includes the development of a strain of Propionibacterium acnes. In some embodiments, the dysbiotic event is psoriasis and the microbiota imbalance includes the growth of bacteria considered not beneficial to the skin. In some embodiments, the dysbiotic event is hidradenitis suppurativa or Verneuil's disease and the imbalance of the microbiota includes the growth of bacteria and a greater diversity of bacteria than in the normal microbiota. In some embodiments, the dysbiotic event is atopic dermatitis and the microbiota imbalance includes growth of Staphylococcus aureus and less bacterial diversity than in the normal microbiota.

In some embodiments, the dysbiotic event is the occurrence of tender skin, irritation, itching, redness, tightness, and/or dry skin. Preferably, the occurrence of sensitive skin, irritation, itching, redness, tightness and/or skin dryness is not pathological or associated with skin pathology.

The term “Fructo-oligosaccharides” or “FOS” denotes oligomers having a maximum degree of polymerization of 10, composed of D-fructoses linked by beta-1,2 bonds and which may contain a molecule of glucose at the one of the ends. FOS are part of the fructans. They are present in nature in a number of plants such as onion, chicory, artichoke, garlic, banana, asparagus and beetroot. They can also be produced industrially by two different processes: by partial hydrolysis of chicory inulin by a specific endoinulinase (inulin being a linear polymer of fructose linked in beta-2,1 with at one end a glucose linked in alpha1-beta2) or by enzymatic synthesis.

By “short-chain Fructo-oligosaccharides” or “scFOS”, is meant fructo-oligosaccharides consisting of 2 to 4 molecules of fructose and containing a molecule of glucose at one of the oligosaccharide extremities. In one embodiment, the short chain fructo-oligosaccharides are derived from sugar beet, wheat, barley, rye, cane, asparagus, banana, artichoke, garlic and/or onion. In one embodiment, the fructo-oligosaccharides are derived from sugar beet. Preferably, they are fructo-oligosaccharides marketed under the trade name Actilight® or FOSbeauty®.

“GF2”, “GF3” and “GF4” denote the names given respectively to 1-kestose, to nystose and to 1-beta-fructofuranosylnystose. The initial letter G corresponds to a unit of glucose and the letter F corresponds to a unit of fructose.

The term “skin microbiota” or “cutaneous microbiota” denotes the community of opportunistic microorganisms, saprobionts, often symbionts and sometimes pathogens which make up the flora present on the surface of the skin, skin appendages and/or mucous membranes. Preferably, the cutaneous microbiota is the microbiota present on the surface of the skin and/or the skin appendages. In one embodiment, the skin microbiota is the microbiota present on the surface of the skin. In another embodiment, the skin microbiota is the microbiota present on the surface of the skin appendages. In a final embodiment, the skin microbiota is the microbiota present on the surface of the mucous membranes. Preferably, the skin microbiota is human skin microbiota.

The term “skin appendages” denotes the integumentary productions originating from the ectoderm and characterized by a high rate of keratinization. In one embodiment, the skin appendages are hair, body hair and/or nails. Preferably, the skin appendages are the hair.

The term “cosmetically acceptable carrier” denotes any adjuvant or excipient allowing the manufacture, storage and/or administration of the cosmetic composition. Preferably, it is a medium which is compatible with the skin, appendages and/or mucous membranes, having a pleasant colour, smell and feel, and not causing any unacceptable discomfort (tingling, tightness or redness in particular) that may discourage the consumer from using the cosmetic composition.

### Cosmetic Method

The present invention relates to a cosmetic method for balancing the skin microbiota. Balancing the skin microbiota notably improves the appearance and/or skin comfort.

The cosmetic method according to the invention comprises the application of at least one short-chain fructo-oligosaccharide to the skin, the skin appendages and/or the mucous membranes. In particular, the at least one short-chain fructo-oligosaccharide is included in a cosmetic composition further comprising a cosmetically acceptable carrier.

The cosmetic method according to the invention makes it possible to improve the aesthetics and/or the comfort of the skin, the skin appendages and/or the mucous membranes on which the composition is applied. It can allow in particular the reduction of inconveniences such as redness, tightness, dull skin and/or itching. The effects obtained are only on the surface of the skin, appendages and/or mucous membranes, and are only cosmetic and non-therapeutic. Thus, the cosmetic method according to the invention is a non-therapeutic method in that it does not make it possible to treat or reduce the symptoms of a pathology, but only to improve the appearance and/or the comfort of the skin, skin appendages and/or mucous membranes by helping to maintain, preserve and/or restore the balance of the skin microbiota.

Preferably, the application is an application to the skin and/or the skin appendages, or topical application.

[ ] The application of the cosmetic composition can be carried out by any suitable method known in the art. The application can be carried out by means of an applicator, such as a foam, a flocked or non-flocked tip, a felt, a brush, a comb, a paintbrush, a woven or a non-woven tissue. Alternatively, the cosmetic composition can be applied in the implementation of the method according to the invention without an applicator, in particular with one or more fingers.

The application can be implemented for any duration and at any suitable frequency. The duration and/or frequency of application may in particular be adapted according to the zone of skin, of skin appendages and/or of the mucous membrane concerned, the surface of the skin, of the appendages and/or of the mucous membranes concerned, the appearance of the skin, the appendages and/or the mucous membranes, and/or the state of the skin microbiota, in particular its imbalance, before applying the cosmetic composition.

The cosmetic composition can be applied for any time sufficient to maintain, restore and/or balance the skin microbiota. The cosmetic composition can be applied for any time sufficient to improve the appearance and/or the comfort of the skin, the skin appendages and/or the mucous membranes. The application can thus be carried out for a period of between 1 day and 6 months, preferably between 2 days and 1 month, in particular between 7 days and 21 days.

The frequency of application of the cosmetic composition in the method according to the invention can be any frequency sufficient to maintain, restore and/or balance the skin microbiota. The frequency of application of the cosmetic composition in the method according to the invention can be any frequency sufficient to improve the appearance and/or the comfort of the skin, of the skin appendages and/or of the mucous membranes. The application can thus be implemented three times a day, twice a day, once a day, once every two days or once a week for example.

In some embodiments, the application in the method of the invention can be a single application, for example following the appearance of at least one sign chosen for example from the group consisting of redness, tightness, dry skin, sensitive skin, itching, irritation and dull skin.

In the method according to the invention, the application of the cosmetic composition can be carried out at any time of the day. In one embodiment, the application is carried out in the morning. In one embodiment, the application is carried out in the evening before going to bed.

The invention further relates to the use of an effective amount of at least one short-chain fructo-oligosaccharide in a cosmetic composition, the composition being intended to balance the skin microbiota.

A further object of the invention is the use of a cosmetic composition comprising, in a cosmetically acceptable carrier, at least one short-chain fructo-oligosaccharide to balance the skin microbiota.

### Cosmetic Composition

The present invention also relates to a cosmetic composition comprising at least one short-chain fructo-oligosaccharide and at least one cosmetically acceptable carrier.

The at least one short-chain fructo-oligosaccharide can be included in the cosmetic composition according to the invention in any suitable amount. In particular, it can be included in the cosmetic composition in an effective amount to improve the appearance and/or the comfort of the skin by helping to restore, maintain and/or preserve the balance of the skin microbiota.

In one embodiment, the at least one short-chain fructo-oligosaccharide is included in the cosmetic composition in an amount ranging from 0.1% to 50% by weight relative to the total volume of the cosmetic composition. In one embodiment, the at least one short-chain fructo-oligosaccharide is included in the cosmetic composition in an amount ranging from 1 to 5% by weight relative to the total volume of the cosmetic composition. In one embodiment, the at least one short-chain fructo-oligosaccharide is included in the cosmetic composition in an amount ranging from 5 to 50%, preferably from 5 to 20%, in particular from 5 to 15% by weight relative to the total volume of the cosmetic composition. In one embodiment, the at least one short-chain fructo-oligosaccharide is included in the cosmetic composition in an amount strictly less than 2.5% by weight relative to the total volume of the cosmetic composition. In one embodiment, the at least one short-chain fructo-oligosaccharide is included in the cosmetic composition in an amount strictly greater than 2.5% by weight relative to the total volume of the cosmetic composition. In one embodiment, the at least one short-chain fructo-oligosaccharide is included in the cosmetic composition in an amount of approximately 5% by weight relative to the total volume of the cosmetic composition.

In one embodiment, the at least one short-chain fructo-oligosaccharide is included in the cosmetic composition in an amount of at least 0.5% by weight relative to the total weight of the cosmetic composition. In one embodiment, the at least one short-chain fructo-oligosaccharide is included in the cosmetic composition in an amount of 0.5 to 5% by weight relative to the total weight of the cosmetic composition. In one embodiment, the at least one short-chain fructo-oligosaccharide is included in the cosmetic composition in an amount of approximately 0.5% by weight relative to the total weight of the cosmetic composition.

In a preferred embodiment, the at least one short-chain fructo-oligosaccharide is included in the cosmetic composition in an amount of approximately 1% by weight relative to the total weight of the cosmetic composition.

In one embodiment, the at least one short-chain fructo-oligosaccharide is included in the cosmetic composition in an amount of approximately 2.5% by weight relative to the total weight of the cosmetic composition.

In one embodiment, the at least one short-chain fructo-oligosaccharide is included in the cosmetic composition in an amount of approximately 5.0% by weight relative to the total weight of the cosmetic composition.

In one embodiment, the at least one short-chain fructo-oligosaccharide is a mixture of at least two, preferably at least three, short-chain fructo-oligosaccharides.

According to one embodiment, the at least one short-chain fructo-oligosaccharide is a mixture of:
a fructo-oligosaccharide containing one glucose unit and two fructose units, also called 1-kestose or GF2, with a degree of polymerization equal to 3;
a fructo-oligosaccharide containing one glucose unit and three fructose units, also called nystose or GF3, with a degree of polymerization equal to 4; And
a fructo-oligosaccharide containing one glucose unit and four fructose units, also called 1-beta-fructofuranosylnystose or GF4, with a degree of polymerization equal to 5.

According to a preferred characteristic, the fructose units of the fructo-oligosaccharides GF2, GF3 and GF4 are linked together by beta(2-1) bonds and the terminal glucose unit is linked to a fructose unit by a alpha1-beta2 bond.

According to one embodiment, the at least one short-chain fructo-oligosaccharide is obtained by an enzymatic reaction, preferably involving a beta-fructofuranoside on sucrose. According to an advantageous characteristic, said sucrose is derived from sugar beet. The product obtained at the end of the enzymatic reaction of sucrose molecules, is a mixture of sucrose, glucose and short-chain fructo-oligosaccharides. Advantageously, this product is purified by chromatography.

According to one embodiment, the at least one short-chain fructo-oligosaccharide comprises between 30 and 45% of GF2, between 40 and 60% of GF3, between 5 and 15% of GF4 and between 1 and 10% of mono- and disaccharides (glucose and sucrose), preferably between 35 and 40% GF2, between 40 and 55% GF3, between 7 and 12% GF4 and between 3 and 8% mono- and disaccharides (glucose and sucrose), more preferably approximately 37% of GF2, approximately 53% of GF3, approximately 10% of GF4 and less than 7% of mono- and disaccharides (glucose and sucrose), the percentages being expressed by weight relative to the total weight of the mixture of fructo-oligosaccharides.

According to a preferred embodiment, the at least one short-chain fructo-oligosaccharide is the product marketed under the brand name ACTILIGHT® by the company TEREOS®. More preferably, the at least one short-chain fructo-oligosaccharide is the product marketed under the brand name ACTILIGHT® 950P, ACTILIGHT® 950S or ACTILIGHT® 951S by the company TEREOS®. According to a second preferred embodiment, the at least one short-chain fructo-oligosaccharide is the product marketed under the brand name FOSbeauty® by the company TEREOS®.

The cosmetic composition according to the invention may be in any of the forms normally used for topical application, in particular in hydroalcoholic form, of an oil-in-water or water-in-oil or multiple emulsion, of an oily gel, or a liquid, pasty or solid anhydrous product or in the form of a dispersion in the presence of spherules. These compositions are prepared according to the usual methods.

The cosmetic composition can be in all the galenic forms which can be envisaged. The composition may take the form of an aqueous, alcoholic, aqueous-alcoholic or oily solution, of a dispersion of the lotion or serum type, of a suspension, of microcapsules or microparticles; vesicular dispersions of the ionic and/or non-ionic type, of an aqueous, oily lotion or in the form of a serum; a foam, a solid preparation, for example a stick; of a composition for aerosol also comprising a propellant under pressure, of a gel, or in the form of a patch.

This composition can be more or less fluid and have the appearance of a white or colored cream, an ointment, a milk, or a paste. It may optionally be applied to the skin, its appendages and/or the mucous membranes in the form of an aerosol.

The cosmetic composition according to the invention may in particular be in the form of a dermo-cosmetic product, a hair product, an intimate hygiene product, a dressing and/or a healing product. In one embodiment, the cosmetic composition according to the invention is in the form of a dermo-cosmetic product, a hair product, an intimate hygiene product and/or a dressing. In one embodiment, the cosmetic composition according to the invention is in the form of a dermo-cosmetic product, a hair product and/or an intimate hygiene product.

The composition according to the invention may be in the form of a cleansing, protective or care composition for the face, or for the body (for example day cream, night cream, make-up remover cream, body protection or care such as moisturizer or sunscreen, lotion, gel or foam for skin care), a make-up composition such as foundation.

The composition according to the invention may be in the form of a hair product such as a shampoo, in particular a solid shampoo, a conditioner, a coloring product, a hair mask, or a styling product.

In one embodiment, the composition according to the invention is chosen from the group consisting of a moisturizer, a sunscreen and a solid shampoo.

When the composition is an emulsion, the proportion of the fatty phase can range from 5% to 80% by weight, and preferably from 5% to 50% by weight relative to the total weight of the composition. The oils, waxes, emulsifiers and co-emulsifiers used in the composition in emulsion form are chosen from those conventionally used in the field of cosmetics.

In known manner, the cosmetic composition according to the invention may also contain the usual adjuvants in the cosmetic and dermatological fields, such as hydrophilic or lipophilic gelling agents, hydrophilic or lipophilic active agents, preservatives, antioxidants, solvents, perfumes, fillers, filters, pigments, chelating agents, odor absorbers and colorants. The amounts of these various adjuvants are those conventionally used in the fields considered, and for example from 0.01% to 20% of the total weight of the composition. These adjuvants, depending on their nature, can be introduced into the fatty phase, into the aqueous phase, into the lipid vesicles and/or into the nanoparticles.

It is possible to use in the compositions of the invention, the at least one short-chain fructo-oligosaccharide in association with at least one further product beneficial for the appearance and/or skin comfort, in particular at least one other product contributing to restore, maintain and/or preserve the balance of the cutaneous microbiota. In certain embodiments, the at least one further product beneficial for appearance and/or skin comfort is chosen from the group consisting of probiotics, resistant dextrins and other prebiotic fibers.

The cosmetic composition according to the invention may thus comprise, in combination with the at least one short-chain fructo-oligosaccharide, at least one native starch.

The at least one native starch may be present in the cosmetic composition according to the invention in any suitable amount. The at least one native starch may be present in the cosmetic composition according to the invention in an amount ranging from 0.5 to 20% by weight relative to the weight of the total composition, in particular from 4.5 to 15% by weight per relative to the total weight of the composition. In the case where the composition according to the invention is in the form of a cream, the at least one native starch is preferably present in an amount ranging from 1 to 10% by weight relative to the total weight of the composition. In certain embodiments, the at least one native starch is present in an amount of 4.5%, 5% or 7% by weight relative to the total weight of the composition. In the case where the composition according to the invention is in the form of a solid, the at least one native starch is preferably present in an amount ranging from 10 to 15% by weight relative to the total weight of the composition. In certain embodiments, the at least one native starch is present in an amount ranging from 10% or 15% by weight relative to the total weight of the composition.

In one embodiment, the cosmetic composition according to the invention comprises:
at least 0.5%, in particular from 0.5 to 5%, by weight relative to the total weight of the composition, of at least one short-chain fructo-oligosaccharide, and
from 0.5 to 20%, in particular from 4.5 to 15%, by weight relative to the total weight of the composition, of at least one native starch, preferably of at least one native corn starch.

The present application thus also relates to cosmetic compositions comprising at least one native starch. Preferably, the native starch is derived from a source of starch chosen from corn, tapioca, wheat, barley, potato or banana. Preferably, the native starch is native corn starch. More preferably, the native starch is a product marketed under the brand name Meritena® by the company TEREOS®, even more preferably it is the product marketed under the brand name Meritena® 100 by the company TEREOS®. The Applicants have indeed demonstrated that the inclusion of native starches in cosmetic compositions according to the invention makes it possible to preserve, or even improve, the sensory and physical properties of the cosmetic compositions. In particular, the combination of at least one native starch with at least one short-chain fructo-oligosaccharide in the cosmetic compositions according to the invention makes it possible to obtain cosmetic compositions, in particular in the form of moisturizers, sunscreens and/or or solid shampoos, the physical and sensory properties of which are improved compared to those of the compositions not comprising such a combination and which make it possible to improve the appearance and/or the comfort of the skin.

In certain embodiments, the at least one short-chain fructo-oligosaccharide is the only compound of the cosmetic composition contributing to restoring, maintaining and/or preserving the balance of the skin microbiota, in particular the at least one short-chain fructo-oligosaccharide is the only compound of the cosmetic composition improving the appearance and/or the comfort of the skin.

### EXAMPLES

The present invention will be better understood on reading the following examples which illustrate the invention without limitation.

### Example 1: Study of the Effect of scFOS—ACTILIGHT®—on Different Bacterial Strains

The present study evaluated the effects of short-chain fructo-oligosaccharides (scFOS, Actilight® P95, Beghin-Meiji) on the in vitro growth and the competitive activity of 3 bacterial strains (Staphylococcus epidermidis, Cutibacterium acnes and Staphylococcus aureus) representative of the human skin microbiota.

### Material and Method

### Bacterial Strains

Staphylococcus epidermidis (ATCC® 12228) and Staphylococcus aureus (ATCC® 6538) were acquired from the American Type Culture Collection (ATCC), while Cutibacterium acnes (CCUG 1794T) was acquired from the Culture Collection University of Gothenburg (CCGU). The bacterial strains were provided in a lyophilized format and reactivated according to the indications of the respective cell bank. Briefly, the entire pellet was resuspended with 0.5 ml of an appropriate growth medium (tryptic soy broth (TSB) for Staphylococcus strains and TSB+5% defibrinated sheep blood for C. acnes (BTSB)). Once rehydrated, the suspensions obtained were inoculated into sterile tubes containing the strain-specific growth medium and, after mixing, incubated under the appropriate conditions (i.e. 24 h at 37° C. under aerobic conditions for Staphylococcus strains and 48 h at 37° C. in anaerobic condition for C. acnes). After 24 hours, the Staphylococcus strain suspensions were streaked on a selective mannitol salt agar plate for staphylococci, while the C. acnes suspension was streaked on agar plates for fastidious bacteria after 48 h of incubation. After 24 h and 48 h of incubation under appropriate conditions, a single colony for each bacterial strain, taken with a sterile inoculation loop, was inoculated into TSB and BTSB liquid medium. When the three bacterial strains have reached their mid-log growth phase (24 h for Staphylococcus strains and 48 h for C. acnes), the concentration of bacteria (bacterial load), in colony-forming units per mL (CFU/mL), present in the inocula, was determined by densitometry, followed by CFU counts after plating on agar of serial bacterial dilutions. Inocula of fresh bacterial strains were prepared before each experiment to ensure treatment consistency.

### Preparation of Actilight®

Actilight® was prepared in accordance with Rossi et al., 2005 (10). Briefly, depending on the liquid medium in which the experiments were performed, the scFOS was weighed and dissolved in both TSB and minimal medium (0.9% NaCl in sterile water+0.003% broth of tryptic phosphate (TPB) 1) at a concentration of 20% (w/v). Once the scFOS were completely dissolved, sterilization of the medium was performed by autoclaving (121° C. for 20 min).

Bacteriostatic and Bactericidal Activity of scFOS

The capacity of scFOS to inhibit the growth of bacterial strains and/or to kill them was evaluated with a standard protocol for the definition of the minimum inhibitory concentration (MIC) and the minimum bactericidal concentration (MBC). The MIC is defined as the lowest concentration of an agent that prevents the visible growth of bacteria, while the MBC is the lowest concentration of an antibacterial agent needed to kill a bacterium over a fixed period (usually 8 h and 24 h). Briefly, 10×10 6 CFU/mL of each bacterial strain were exposed to an increasing concentration of scFOS (from 0 to 15% (w/v)), in liquid broth medium (TSB for S. aureus and S. epidermidis, and BTSB for C. acnes) for 24 h at 37° C., with constant shaking under aerobic conditions. The impact of different concentrations of scFOS on the growth of bacterial strains was followed with a spread plate approach. Aliquots of the inocula were collected at 0, 8 and 24 h, serially diluted and seeded on specific agar plates (mannitol salt agar plates for Staphylococcus strains and blood agar for C. acnes). After 24 h (S. aureus and S. epidermidis) and 48 h (C. acnes), the visible colonies, formed on the spread plates, were counted. When corrected for the appropriate dilution factor, bacterial growth was calculated as a fold change from control (t=0 h).

Impact of scFOS on the Growth Kinetics of Bacterial Strains

The capacity of the bacterial strains tested to use scFOS as an energy source, to support their growth, was explored by exposing them to different concentrations of scFOS in a minimal liquid medium. Briefly, following centrifugation and thorough washing to remove the growth medium in which they were inoculated, the 3 bacteria (10×10⁶ CFU/mL) were exposed to increasing concentrations of scFOS (from 0 to 15%) in minimal medium at 37° C. for 24 h, under constant shaking and under aerobic conditions. As described above, the impact of different concentrations of scFOS on the growth of bacterial strains was assessed with a spread plate approach. Aliquots of the inocula were collected at 8 and 24 h, serially diluted and seeded on specific agar plates (mannitol salt agar plates for Staphylococcus strains and blood agar for C. acnes). After 48 h at 37° C. under aerobic conditions, the colonies formed on the different spread plates were counted. Once corrected by the appropriate dilution factor, the bacterial growth of the 3 strains tested was calculated as a factor of change with respect to the bacterial load of the initial inoculum (t=0 h).

Competition Between Strains of Bacteria for scFOS

The ability of bacterial strains to compete for scFOS as an energy source was evaluated through comparisons of S. epidermidis vs. C. acnes and S. epidermidis vs. S. aureus. These competitions were carried out at different concentrations of scFOS (from 0 to 5%) in a minimal medium. The evolution of competition was assessed with a spread plate approach, based on selective means and differential colony counting. Briefly, after centrifugation and thorough washing to remove growth medium, 10×10⁶ CFU/mL of each competing strain were exposed to increasing concentrations of scFOS (from 0 to 5%) in minimal medium (0.9% NaCl in sterile water+0.003% TPB) at 37° C. for 48 h, with constant stirring and under aerobic conditions. Aliquots of the inocula were taken at 0 (initial bacterial load), 4, 8, 24 and 48 h, serially diluted and seeded on specific agar plates. For S. epidermidis vs. S. aureus competition, inoculation was performed on mannitol phenol red salt agar (MSARP).

For the S. epidermidis vs. C. acnes competition, a differential colony counting strategy was applied. Briefly, the same aliquots, collected at different times, were seeded onto MSARP and blood agar plates. The number of CFU/mL of C. acnes was calculated by subtracting the number of colonies obtained from MSARP plates, specific for S. epidermidis, from that of blood agar.

Once corrected by the appropriate dilution factor, the growth of the competing bacterial strains was calculated as a variation factor with respect to the bacterial load of the initial inoculum (t=0 h).

### Statistical Analyzes

All the statistical analyzes were carried out with the OriginLab software. To determine if statistically significant differences between treatments were present, a t-test analysis was performed. All data are presented as the mean±standard deviation (SD) of 3 independent experiments. Differences between groups were considered significant at p<0.05.

### Results

Impact of scFOS on Bacterial Growth and Survival

Before studying the potential effect of scFOS as a prebiotic on their growth, their impact on the growth and survival of S. epidermidis, C. acnes and S. aureus was tested. The experiments were carried out in bacterial growth medium specific for each bacterial strain (TSB for S. aureus and S. epidermidis and BTSB for C. acnes).

As shown in Table 1, no bacteriostatic or bactericidal effect of scFOS was observed on S. epidermidis, C. acnes and S. aureus, since a significant growth compared to the initial bacterial load was put evident at all tested concentrations of scFOS. A decrease in the populations of S. epidermidis and S. aureus, between 8 and 24 h at the same concentration was observed, probably due to the progressive depletion of nutrients, limiting the survival of the bacteria. A significant reduction in the growth of C. acnes and S. aureus was observed at the highest scFOS concentrations (10 and 15% scFOS).

TABLE 1
Bacteriostatic or bactericidal effect of scFOS on S. epidermidis, C. acnes and S. aureus.
Prebiotic activity of scFOS on bacterial strains

| scFOS | S. epidermidis | | | C. acnes | | | S. aureus | | |
|---|---|---|---|---|---|---|---|---|---|
| (%) | 0 h | 8 h | 24 h | 0 h | 8 h | 24 h | 0 h | 8 h | 24 h |
| 0 | 1.0 ± 0.1 | 26.7 ± 0.5 | 23.7 ± 6.3 | 1.0 ± 0.1 | 11.7 ± 1.0 | 0.3 ± 0.5 | 1.0 ± 0.2 | 262.9 ± 11.5 | 141.5 ± 30.1 |
| 1 | 1.0 ± 0.1 | 46.6 ± 4.6 | 15.2 ± 0.3 | 1.0 ± 0.1 | 10.5 ± 0.3 | 2.6 ± 0.2 | 1.0 ± 0.2 | 293.2 ± 36.2 | 196.4 ± 25.9 |
| 5 | 1.0 ± 0.1 | 45.2 ± 4.4 | 9.2 ± 0.2 | 1.0 ± 0.1 | 9.6 ± 1.3 | 1.0 ± 0.5 | 1.0 ± 0.2 | 227.6 ± 35.0 | 154.3 ± 32.5 |
| 10 | 1.0 ± 0.1 | 54.1 ± 9.3 | 3.4 ± 0.2 | 1.0 ± 0.1 | 2.9 ± 0.7 | 2.9 ± 2.2 | 1.0 ± 0.2 | 116.3 ± 22.3 | 82.7 ± 9.6 |
| 15 | 1.0 ± 0.1 | 63.5 ± 19.0 | 3.4 ± 0.6 | 1.0 ± 0.1 | 1.6 ± 0.1 | 0.3 ± 0.1 | 1.0 ± 0.2 | 130.4 ± 4.8 | 28.1 ± 19.3 |

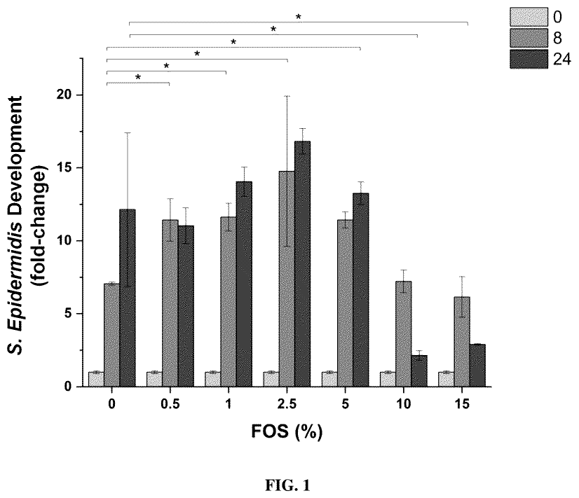

In minimal medium, bacterial growth was significantly limited in the absence of scFOS (0%), to ensure that bacterial growth was directly dependent on scFOS metabolization. As shown in FIG. 1, S. epidermidis was able to exploit scFOS as an energy source. Indeed, its bacterial load increased significantly after 8 h from 0.5 to 5% scFOS and tended to increase up to 24 h, in the presence of 1 and 5% scFOS. However, higher concentrations of scFOS (10 and 15%) negatively affected its growth compared to the control (0% scFOS).

Unlike S. epidermidis, S. aureus and C. acnes have not been able to exploit scFOS as an energy source because the growth of these bacterial strains has always been significantly lower than that in the absence of scFOS, independently of the concentrations and the time tested. However, while the S. aureus population was still able to increase under these conditions, the growth of the C. acnes population was completely halted. A significant decrease in the population of the two bacteria from 8 to 24 h was observed, probably due to the progressive depletion of the few nutrients available in the minimal medium which, in turn, led to the death of the bacteria (FIG. 1).

Competition of Bacterial Strains for scFOS in Minimal Medium

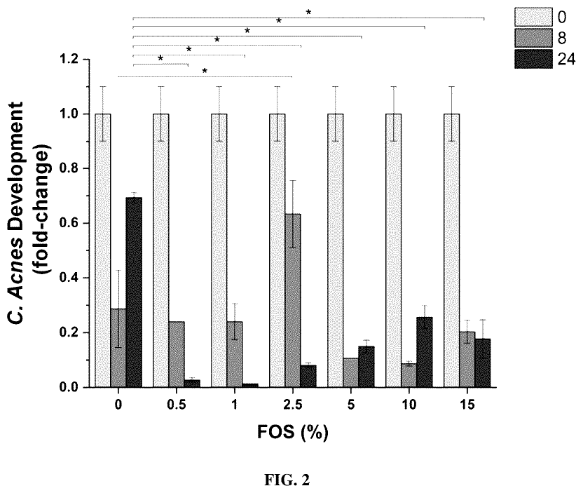

Competition for scFOS between S. epidermidis and C. acnes in minimal medium confirmed the results observed in the previous section. Indeed, while no positive effect of scFOS on the growth of the population of C. acnes was demonstrated at 8 h, the presence of scFOS stimulated the growth of S. epidermidis at all the doses tested up to 5% scFOS (FIG. 2). This results in a positive growth ratio of S. epidermidis/C. acnes at 8 h, with a maximum reached at the dose of 1% scFOS (Table 2).

TABLE 2
S. epidermidis/C. acnes and S. epidermidis/S. aureus growth ratios.

| | |
|---|---|
| S. epidermidis/ | S. epidermidis/ |
| C. acnes | S. aureus |
| Growth Ratio | Growth Ratio |

| scFOS (%) | 8 h | 24 h | 8 h | 24 h |
|---|---|---|---|---|
| 0 | 1.1 | — | 1.6 | 2.0 |
| 0.5 | 7.8 | — | 2.8 | 5.0 |
| 1 | 12.8 | — | 1.8 | — |
| 2.5 | 8.5 | — | 2.5 | 6.0 |
| 5 | 3.4 | — | 1.6 | 2.0 |

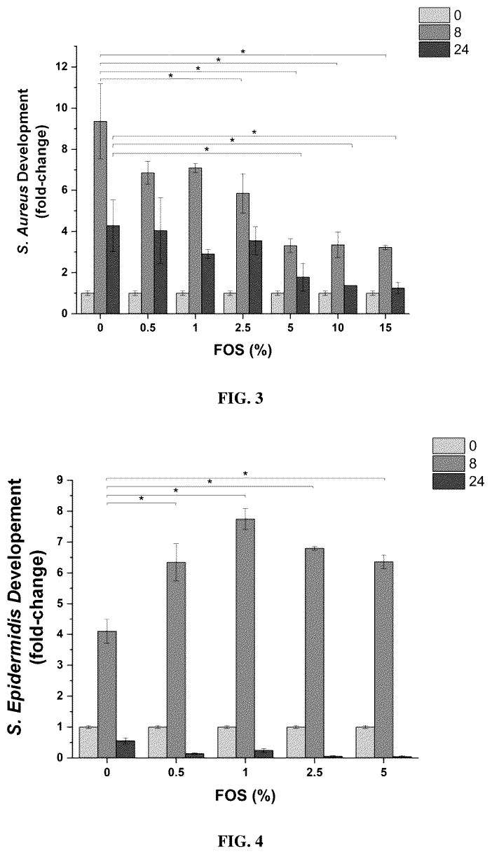

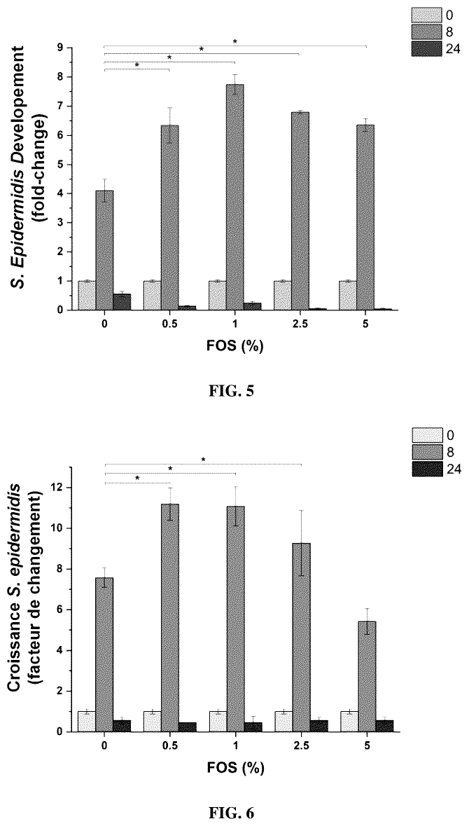

The competition between S. epidermidis and S. aureus was characterized by results similar to those of the competition tested previously, without positive effect on the growth of S. aureus while the growth of S. epidermidis was stimulated by an addition of scFOS at a dose of between 0.5 and 2.5% after 8 h of culture in minimal medium (FIG. 3). The calculated ratio of S. epidermidis to S. aureus growth confirmed the ability of S. epidermidis to grow using scFOS at the expense of S. aureus growth, with the highest stimulation achieved at the lowest dose. of scFOS (0.5%) (Table 2).

### Example 2: Composition of Moisturizing Cream According to the Invention

A moisturizing cream composition according to the invention is described in Table 3. Three alternatives of this composition were prepared, depending on whether native corn, wheat or potato starch was incorporated.

TABLE 3
Composition of moisturizing cream according to the invention.

| Phase | Ingredients | % in the formulation |
|---|---|---|
| A (Aqueous) | Glycerine | 5.0 |
| | Native starch | 5.0 (Corn) or |
| | | 7.0 (wheat) or |
| | | 7.0 (potato) |
| | Actilight ® 950 P | 1.0 |
| | Water | QSP 100 |
| B (Lipid) | Isopropyl palmitate | 5.0 |
| | Cetearyl alcohol | 4.0 |
| | Shea butter | 3.0 |
| | Vegetal oil | 2.0 |
| | Cetyl Palmitate | 1.0 |
| | Vitamin E | 0.1 |
| C (preservartive) | Phenoxyethanol | 0.2 |

The percentages correspond to percentages by weight relative to the weight of the composition.

The composition according to Table 3 was prepared by the following process:
Weighing of phases A, B and C,
Heating phase A to 85° C. for up to 45 minutes with continuous stirring,
Heating of phase B to 75° C.,
Slow incorporation of phase C, then of phase B, into phase A while maintaining agitation,
Stirring until the emulsion is stabilized.

A white cream with a shiny texture was obtained.

### Example 3: Composition of Sunscreen According to the Invention

A sunscreen composition according to the invention is described in Table 4.

TABLE 4
Composition of sunscreen according to the invention.

| Phase | Ingredients | % in the formulation |
|---|---|---|
| A | Micronized Zinc oxide | 24.4 |
| (Aqueous) | Glycerin | 5.0 |
| | Calendula hydrosol | 2.0 |
| | Corn Starch | 4.5 |
| | Actilight ® 950 P | 1.0 |
| | Water | 47.8 |
| B | Isopropyl Palmitate | 7.0 |
| (Lipid) | Cetearyl Alcohol | 4.0 |
| | Shea Butter | 2.0 |
| | Vegetal Oil | 2.0 |
| | Beeswax | 2.0 |
| | Vitamin E | 0.1 |
| C (preservative) | Phenoxyethanol | 0.2 |

The percentages correspond to percentages by weight relative to the weight of the composition.

The composition according to Table 4 was prepared by the following process:
Weighing of phases A, B and C,
Heating of phase A to 85° C.,
Heating of phase B to 75-80° C.,
Slow incorporation of phase C, then of phase B, into phase A while maintaining agitation,
Stirring until the emulsion is stabilized.

### Example 4: Solid Shampoo Composition According to the Invention

A solid shampoo composition according to the invention is described in Table 5.

TABLE 5
Solid shampoo composition according to the invention.

| Phase | Ingredients | % in the formulation |
|---|---|---|
| A | Sodium cocoyl isethionate | 50.0 |
| (Aqueous) | Water | 21.0 |
| | Corn starch | 15.0 |
| | Actilight ® 950 P | 1.0 |
| | Ylang-ylang hydrosol | 4.0 |
| | Damask rose hydrosol | 4.0 |
| B | Shea Butter | 3.0 |
| (Lipid) | Jojoba oil | 2.0 |

The percentages correspond to percentages by weight relative to the weight of the composition.

The composition according to Table 5 was prepared by the following process:
Weighing of phase B and heating to 65° C.,
Gently mix water and surfactant (sodium cocoyl isethionate) in a bowl with a spatula,
Addition of native corn starch when the dough is smooth,
Addition of phase B when the paste is homogeneous,
Mix until the dough is smooth, then pour into molds
Drying of the bars for one day in the moulds, then for 2 days outside the molds at room temperature.

### Example 5: Characterization of the Compositions According to the Invention

### A-Sensory Characterization

The sensory characterization of the compositions according to the invention was carried out by groups of 7 to 8 people.

Sensory Characterization of Moisturizing Creams According to the Invention

The composition of the moisturizing creams evaluated corresponds to that of the moisturizing cream in Table 3, in which the amount of Actilight® 950 P was varied from 0.5% to 5%.

Table 6 summarizes the characteristics of the moisturizing creams according to the invention. The values indicated are averages for the entire workforce. The reference composition is a composition comprising all the other ingredients, including 5% native corn starch, but not comprising Actilight® 950 P.

TABLE 6
Characteristics of the moisturizing
creams according to the invention.

| Actilight ® 950 P Quantity | Integrity | Shine | Firmness | Thickness |
|---|---|---|---|---|
| Reference | 3.95 | 2.8 | 2.82 | 1.63 |
| 0.5% | 3.02 | 2.95 | 1.56 | 1.54 |
| 1.0% | 4.46 | 2.09 | 3.01 | 2.77 |
| 2.5% | 3.61 | 3.7 | 1.52 | 1.42 |
| 5.0% | 4.04 | 3.75 | 1.96 | 1.96 |

Sensory Characterization of Sunscreens According to the Invention

The composition of the sunscreens evaluated corresponds to that of the sunscreen in Table 4, in which the amount of Actilight® 950 P was varied from 0.5% to 5%.

Table 7 summarizes the characteristics of the sun creams according to the invention. The values indicated are averages for the entire workforce. The reference composition is a composition comprising all the other ingredients, but not comprising Actilight® 950 P.

TABLE 7
Characteristics of sunscreens according to the invention.

| Actilight ® 950 P Quantity | Integrity |
|---|---|
| Reference | 2.76 |
| 0.5% | 3.31 |
| 1.0% | 4.02 |
| 2.5% | 3.91 |
| 5.0% | 3.83 |

The sun creams according to the invention are more compact than those without the inclusion of short-chain fructo-oligosaccharides.

Sensory Characterization of Solid Shampoos According to the Invention

Table 8 summarizes the characteristics of the solid shampoos according to the invention. The solid shampoo according to the invention comprises 5% by weight of short-chain fructo-oligosaccharides, and corn starch. The reference composition is a composition comprising all the other ingredients, but not comprising Actilight® 950 P. The second reference is a commercial solid shampoo composition.

TABLE 8
Characteristics of the solid shampoos according to the invention.

| | | Commercial | Solid Shampoo |
|---|---|---|---|
| | Reference | Reference | of the invention |
| Appreciation | 4 | 4.5 | 5 |
| Softness during application | 3.5 | 4.5 | 5 |
| Softness after application | 4.5 | 4.5 | 5 |
| Foaming capacity | 4 | 4 | 4 |
| Foam stability | 5 | 5 | 5 |
| Eye irritation and skin cuts | 1 | 3 | 1 |

The solid shampoos according to the invention have demonstrated particularly advantageous properties in terms of sensory feeling during and after their use. In particular, their softness during and after application was positively noticed, as well as their foaming capacity.

### B-Physical Characterization

Physical Characterization of Moisturizing Creams According to the Invention

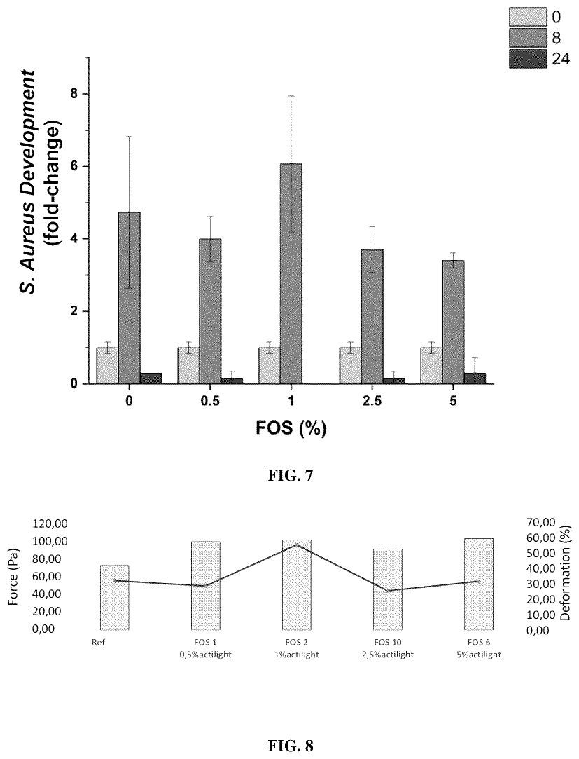

FIG. 8 illustrates the rheology of moisturizers according to the invention. The moisturizing compositions according to the invention have greater elasticity (greater deformation) than the reference which does not comprise a short-chain fructo-oligosaccharide. In addition, the compositions according to the invention have a toughness of the same order of magnitude as that of the reference composition, with the exception of the composition comprising 1% of Actilight® 950 P which has a markedly higher toughness.

FIG. 9 illustrates the viscosity during spreading at 100 s⁻¹ of the moisturizing creams according to the invention. The moisturizing creams according to the invention have a viscosity of the same order of magnitude as that of the reference composition, with the exception of the composition comprising 1% of Actilight® 950 P which has a markedly higher viscosity.

The effect of the incorporation of short-chain fructo-oligosaccharides on the color of the compositions was also measured. Table 9 provides the values of the parameters L*, a* and b* of the moisturizing creams according to the invention, and of two reference creams: the cream not comprising any short-chain fructo-oligosaccharide, and a commercial reference cream.

The parameters L*, a* and b* are the well-known system parameters L*a*b*, and are:
the clarity L* which takes values between 0 (black) to 100 (reference white);
the parameter a* which represents the value on a green→red axis; And
the parameter b* which represents the value on a blue→yellow axis.

TABLE 9
L*, a* and b* parameters of two reference creams and
moisturizing creams according to the invention.

| Actilight ® 950 P Quantity | L* | a* | b* |
|---|---|---|---|
| Reference (0%) | 84.87 | −1.53 | 2.92 |
| Commercial Reference | 87.55 | −3.16 | 7.4 |
| 0.5% | 84.37 | −1.76 | 6.17 |
| 1.0% | 86.76 | −1.58 | 5.9 |
| 2.5% | 83.52 | −1.7 | 5.74 |
| 5.0% | 84.85 | −1.73 | 5.73 |

The incorporation of short-chain fructo-oligosaccharides has a slight impact on the b* parameter, with a slight yellowing of the cream. However, the moisturizing creams according to the invention remain less yellow than the commercial reference.

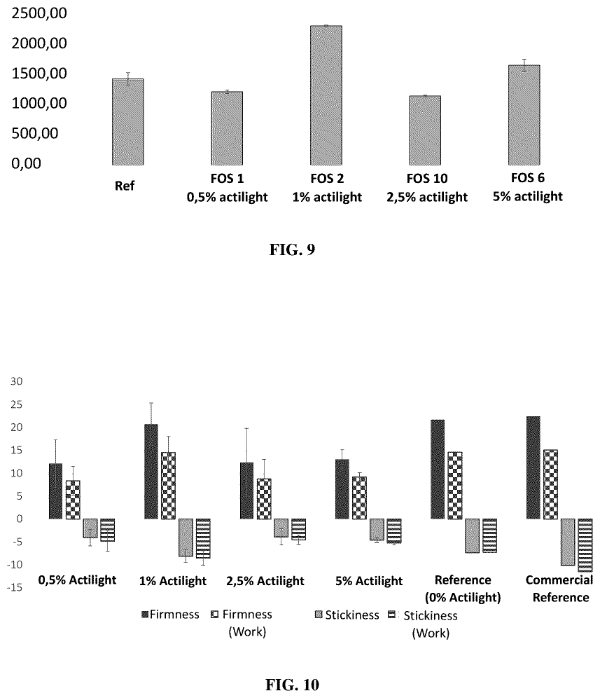

Finally, FIG. 10 illustrates the firmness and the sticky aspect of the moisturizing creams according to the invention, in comparison with those of the reference cream which does not contain short-chain fructo-oligosaccharides, and with those of the reference cream commercial.

The firmness and stickiness of the moisturizing creams according to the invention are of the same order of magnitude as those of the reference creams, both the reference cream which does not comprise short-chain fructo-oligosaccharides and the reference cream commercial.

In conclusion, the incorporation of short-chain fructo-oligosaccharides into the moisturizing creams according to the invention does not alter the physical properties of the creams. The incorporation of short-chain fructo-oligosaccharides in an amount of 1% even seems to improve the physical properties of moisturizers.

Physical Characterization of Sunscreens According to the Invention

FIG. 11 illustrates the rheology of sunscreens according to the invention. The sunscreen compositions according to the invention have greater elasticity (greater deformation) than the reference which does not comprise a short-chain fructo-oligosaccharide. In addition, the compositions according to the invention have a toughness of the same order of magnitude as that of the reference composition.

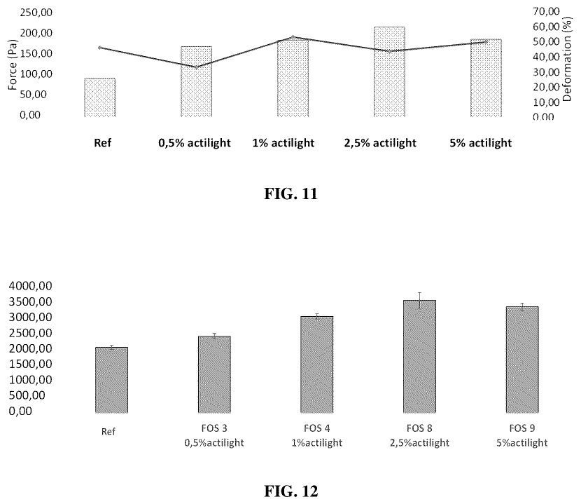

FIG. 12 illustrates the viscosity during spreading at 100 s⁻¹ of the sun creams according to the invention. The sun creams according to the invention have a viscosity which gradually increases with the proportion of short-chain fructo-oligosaccharides, up to 2.5%.

The effect of incorporating short-chain fructo-oligosaccharides on the color of sunscreen compositions was also measured. Table 10 provides the values of the L*, a* and b* parameters of the sun creams according to the invention, and of three reference sun creams: the cream not comprising any short-chain fructo-oligosaccharide, and two sun creams of commercial references.

TABLE 10
Parameters L*, a* and b* of three reference creams
and sunscreens according to the invention.

| Actilight ® 950 P Quantity | L* | a* | b* |
|---|---|---|---|
| Reference (0%) | 92.12 | −0.95 | 3.49 |
| Commercial Reference A | 87.97 | −0.49 | 5.44 |
| Commercial Reference B | 92.52 | −2.52 | 7.29 |
| 0.5% | 92.56 | −0.98 | 3.5 |
| 1.0% | 92.19 | −0.97 | 3.23 |
| 2.5% | 92.41 | −1.01 | 3.22 |
| 5.0% | 91.8 | −0.96 | 3.26 |

The inclusion of short-chain fructo-oligosaccharides has no significant effect on the color of sunscreens.

In conclusion, the inclusion of short-chain fructo-oligosaccharides in sunscreens increases their elasticity and their viscosity, without affecting their color. Elasticity and viscosity are desired properties for such creams, and are often achieved by adding xanthan and/or synthetic polymers, which is not necessary for the creams of the invention.

Physical Characterization of Solid Shampoos According to the Invention

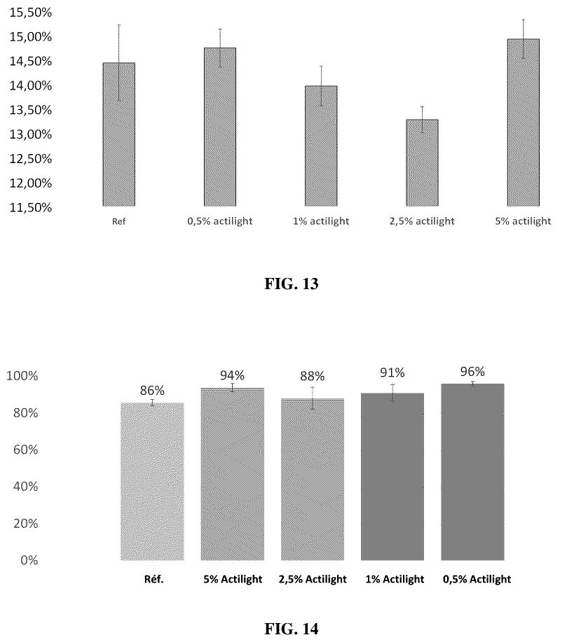

FIG. 13 presents the humidity values in % of the solid shampoos according to the invention. Target moisture values for a solid shampoo are the range of 10% to 20% moisture. All the solid shampoos according to the invention are therefore within the target range of moisture values, regardless of the amount of short-chain fructo-oligosaccharides.

FIG. 14 shows the results of the dissolution test of the solid shampoos according to the invention. The inclusion of short chain fructo-oligosaccharides in solid shampoos seems to slightly improve the dissolution of shampoos.

The effect of the incorporation of short-chain fructo-oligosaccharides on the color of solid shampoos was also measured. Table 11 provides the values of the parameters L*, a* and b* of the solid shampoos according to the invention, and of three reference solid shampoos: the solid shampoo not comprising any short-chain fructo-oligosaccharide, and two solid shampoos commercial references A and B.

TABLE 11
L*, a* and b* parameters of three reference solid shampoos
and solid shampoos according to the invention.

| Actilight ® 950 P Quantity | L* | a* | b* |
|---|---|---|---|
| Reference (0%) | 78.54 | −1.31 | 1.78 |
| Commercial Reference A | 95.8 | −1.58 | 7.67 |
| Commercial Reference B | 71.73 | −1.03 | −0.06 |
| 0.5% | 82.51 | −1.8 | 11.65 |
| 1.0% | 81.38 | −1.71 | 12.03 |
| 2.5% | 76.21 | −2.61 | 10.47 |
| 5.0% | 82.14 | −1.94 | 10.38 |

The inclusion of short-chain fructo-oligosaccharides has no significant effect on the color of solid shampoos.

In conclusion, the solid shampoos according to the invention have suitable moisture values, have a slightly improved dissolution, and their color is not degraded compared to the solid shampoos not comprising short-chain fructo-oligosaccharide.

### Example 6: Effects of the Combination of scFOS with Native Starch

The present analysis evaluates the effects of the combination of short-chain fructo-oligosaccharides (scFOS, Actilight® P95, Beghin-Meiji) with native corn starch (Meritena® 100, TEREOS®) on the properties of texture as well as the sensory properties of cosmetic formulations.

Moisturizing cream compositions according to the invention and comparative compositions are described in Table 12.

TABLE 12
Composition of a moisturizer according to the invention and of four comparative compositions.

| Composition | | % in the formulation | | | | |
|---|---|---|---|---|---|---|
| Phase | Ingredients | Invention | A | B | C | D |
| A | Glycerin | 5.0 | 5.0 | 5.0 | 5.0 | 5.0 |
| (Aqueous) | Native starch | 5.0 | 5.0 | — | — | — |
| | Actilight ® 950 P | 5.0 | — | — | 5.0 | 5.0 |
| | Xanthan | — | — | 4.0 | 4.0 | — |
| | Water | 69.7 | 74.7 | 75.7 | 70.7 | 74.7 |
| B | Isopropyl Palmitate | 5.0 | 5.0 | 5.0 | 5.0 | 5.0 |
| (Lipid) | Cetearyl alcohol | 4.0 | 4.0 | 4.0 | 4.0 | 4.0 |
| | Shea Butter | 3.0 | 3.0 | 3.0 | 3.0 | 3.0 |
| | Vegetable oil | 2.0 | 2.0 | 2.0 | 2.0 | 2.0 |
| | Cetyl Palmitate | 1.0 | 1.0 | 1.0 | 1.0 | 1.0 |
| | Vitamin E | 0.1 | 0.1 | 0.1 | 0.1 | 0.1 |
| C | Phenoxyethanol | 0.2 | 0.2 | 0.2 | 0.2 | 0.2 |
| (Preservative) | | | | | | |

The composition according to Table 12 was prepared by the following process:
Weighing of phases A, B and C,
Heating of phase A to 85° C.,
Heating of phase B to 75° C.,
Slow incorporation of phase C, then of phase B, into phase A while maintaining agitation,
Stirring until the emulsion is stabilized.

The amount of 4% xanthan was retained as the relevant amount of thickener (formulations B and C) for comparison with 5% corn starch (formulation A and formulation according to the invention), in order to compare formulations with similar textures (for example, 5% xanthan would lead to a too viscous formulation).

### Rheological Characterization of Compositions

The rheological properties of the compositions according to table 12 are presented in table 13.

TABLE 13
Rheological properties of the compositions according to example 6.

| | Pour Point at 25° C. | | |
|---|---|---|---|
| | | Resistance | Viscosity |
| | | (Force needed | (mPa · s) at |
| | Elasticity | to break the | spreading |
| Composition | (deformation %) | cream in Pa) | at 100 s⁻¹ |
| Invention | 60.28 | 55.02 | 1621.17 ± 10.69 |
| (scFOS + | | | |
| Starch) | | | |
| A (Starch) | 42.45 | 55.75 | 1401.63 ± 101.04 |
| B (xanthan) | 147.73 | 48.16 | 1288.50 ± 44.27 |
| C (scFOS + | 103.00 | 135.82 | 2228.36 ± 88.58 |
| xanthan) | | | |
| D (scFOS) | 1.22 | 0.04 | 29.1731 ± 3.13 |

The combination of scFOS with a thickening agent increased the viscosity during spreading. Indeed, the combination according to the invention has a higher viscosity during spreading compared to that of composition A comprising only starch. The same goes for the combination of scFOS with xanthan.

However, when the scFOS are associated with starch, the strength of the cream is not impacted. Indeed, the combination of scFOS with native starch according to the invention did not modify the viscosity resistance to composition A comprising only starch.

### Texture Characterization of Compositions

The texture properties by penetrometry at 25° C. of the compositions according to table 12 are presented in table 14.

TABLE 14
Texture properties of compositions according to Table 12.

| | Firmness | Firmness | Stickiness | Stickiness |
|---|---|---|---|---|
| Composition | (g) | (work; g · s) | (g) | (work; g · s) |
| Invention | 9.153 ± 0.48 | 13.018 ± 1.035 | −4.649 ± 0.198 | −5.221 ± 0.235 |
| (scFOS + Starch) | | | | |
| A (Starch) | 14.543 ± 1.203 | 21.702 ± 3.962 | −7.391 ± 0.866 | −7.285 ± 0.326 |
| B (xanthan) | 12.476 ± 0.309 | 18.679 ± 0.73 | −5.79 ± 0.562 | −4.339 ± 0.602 |
| C (scFOS + xanthan) | 14.947 ± 0.499 | 29.774 ± 1.126 | −6.698 ± 0.481 | −4.552 ± 0.511 |
| D (scFOS) | — | — | — | — |

The presence of scFOS in combination with native starch lowered the overall firmness of the formulation, whereas the opposite phenomenon was observed with the combination of scFOS with xanthan.

### Sensory Characterization of Compositions

The sensory characterization of the compositions according to table 12 was carried out by groups of 5 people.

The sensory properties of the compositions according to Table 12 are presented in Table 15.

TABLE 15
Sensory properties of compositions according to Table 12.

| | | Invention | | C |
|---|---|---|---|---|
| | A | (scFOS + | B | (scFOS + |
| | (Starch) | Starch) | (xanthan) | xanthan) |
| Shine | 3.20 ± 0.27 | 4.30 ± 0.67 | 4.20 ± 0.45 | 2.50 ± 0.71 |
| Firmness | 2.50 ± 1.00 | 1.70 ± 0.91 | 3.00 ± 1.41 | 3.60 ± 0.89 |
| Thickness | 2.10 ± 0.89 | 1.20 ± 0.27 | 2.10 ± 0.74 | 2.80 ± 0.84 |
| Greasy effect | 3.00 ± 1.17 | 1.90 ± 0.89 | 3.20 ± 1.10 | 3.00 ± 1.41 |
| Sticky effect | 1.60 ± 0.89 | 1.90 ± 0.74 | 2.70 ± 1.20 | 2.80 ± 1.04 |
| Slippery effect | 3.50 ± 1.00 | 3.00 ± 0.82 | 2.60 ± 1.14 | 2.60 ± 0.55 |
| Absorption | 3.30 ± 0.67 | 3.70 ± 0.45 | 2.00 ± 0.94 | 2.10 ± 1.14 |
| Appreciation | 3.30 ± 0.84 | 3.50 ± 1.12 | 1.60 ± 1.14 | 1.70 ± 1.48 |

The formulation comprising the combination of Actilight with starch was more appreciated compared to the formulation comprising only starch thanks to its less greasy feel and thanks to the better shine provided.

The presence of Actilight did not modify the texture characteristics of the starch-based formulations, but the sensory evaluation showed advantageous modifications.

The formulation based on corn starch and Actilight proved to be better than the other formulations, in particular compared to the formulations based on xanthan.

### REFERENCE LIST

1. Sfriso, R.; Egert, M.; Gempeler, M.; Voegeli, R.; Campiche, R. Revealing the secret life of skin—with the microbiome you never walk alone. Int J Cosmet Sci 2020. 42(2): p. 116-126, DOI: 10.1111/ics.12594.
2. Kong, H. H.; Segre, J. A. Skin microbiome: looking back to move forward. J Invest Dermatol 2012. 132(3 Pt 2): p. 933-9, DOI: 10.1038/jid.2011.417.
3. Sanford, J. A.; Gallo, R. L. Functions of the skin microbiota in health and disease. Semin Immunol 2013. 25(5): p. 370-7, DOI: 10.1016/j.smim.2013.09.005.
4. Krutmann, J. Pre- and probiotics for human skin. J Dermatol Sci 2009. 54(1): p. 1-5, DOI: 10.1016/j.jdermsci.2009.01.002.
5. Grice, E. A.; Segre, J. A. The skin microbiome. Nat Rev Microbiol 2011. 9(4): p. 244-53, DOI: 10.1038/nrmicro2537.
6. Ladizinski, B.; McLean, R.; Lee, K. C.; Elpern, D. J.; Eron, L. The human skin microbiome. Int J Dermatol 2014. 53(9): p. 1177-9, DOI: 10.1111/ijd.12609.
7. Al-Ghazzewi, F. H.; Tester, R. F. Impact of prebiotics and probiotics on skin health. Benef Microbes 2014. 5(2): p. 99-107, DOI: 10.3920/BM2013.0040.
8. Dreno, B.; Araviiskaia, E.; Berardesca, E.; Gontijo, G.; Sanchez Viera, M.; Xiang, L. F.; Martin, R.; Bieber, T. Microbiome in healthy skin, update for dermatologists. J Eur Acad Dermatol Venereol 2016. 30(12): p. 2038-2047, DOI: 10.1111/jdv.13965.
9. Grice, E. A.; Kong, H. H.; Renaud, G.; Young, A. C.; Program, N.C.S.; Bouffard, G. G.; Blakesley, R. W.; Wolfsberg, T. G.; Turner, M. L.; Segre, J. A. A diversity profile of the human skin microbiota. Genome Res 2008. 18(7): p. 1043-50, DOI: 10.1101/gr.075549.107.
10. Rossi, M.; Corradini, C.; Amaretti, A.; Nicolini, M.; Pompei, A.; Zanoni, S.; Matteuzzi, D. Fermentation of fructooligosaccharides and inulin by bifidobacteria: a comparative study of pure and fecal cultures. Appl Environ Microbiol 2005. 71(10): p. 6150-8, DOI: 10.1128/AEM.71.10.6150-6158.2005.

1-15. (canceled)

16. A cosmetic method for balancing the skin microbiota of a subject in need thereof, comprising a step of applying to all or part of the skin, skin appendages and/or mucous membranes of the subject in need thereof a cosmetic composition comprising, in a cosmetically acceptable carrier, at least one fructo-oligosaccharide short chain and at least one native starch.

17. The cosmetic method according to claim 16, for improving the skin appearance and/or skin comfort of the subject in need thereof.

18. The cosmetic method according to claim 17, wherein improving the skin appearance and/or skin comfort of the subject in need thereof comprises reducing at least one skin discomfort chosen from the group consisting of redness, tightness, dull skin and itching.

19. The cosmetic method according to claim 16, wherein the at least one fructo-oligosaccharide is derived from sugar beet.

20. The cosmetic method according to claim 16, wherein the at least one short-chain fructo-oligosaccharide is a mixture of:
a fructo-oligosaccharide containing one glucose unit and two fructose units, also called 1-kestose or GF2, with a degree of polymerization equal to 3;
a fructo-oligosaccharide containing one glucose unit and three fructose units, also called nystose or GF3, with a degree of polymerization equal to 4; and
a fructo-oligosaccharide containing one glucose unit and four fructose units, also called 1-beta-fructofuranosylnystose or GF4, with a degree of polymerization equal to 5.

21. The cosmetic method according to claim 16, wherein the at least one native starch is a native corn starch.

22. The cosmetic method according to claim 16, wherein the at least one native starch is included in the cosmetic composition in an amount ranging from 0.5% to 20% by weight relative to the total weight of the composition.

23. The cosmetic method according to claim 16, wherein the at least one short-chain fructo-oligosaccharide is included in the cosmetic composition in an amount of at least 0.5% by weight relative to the weight total of the cosmetic composition.

24. A cosmetic composition comprising, in a cosmetically acceptable carrier, at least one short-chain fructo-oligosaccharide and at least one native starch, wherein the at least one short-chain fructo-oligosaccharide is a mixture of:
a fructo-oligosaccharide containing one glucose unit and two fructose units, also called 1-kestose or GF2, with a degree of polymerization equal to 3;
a fructo-oligosaccharide containing one glucose unit and three fructose units, also called nystose or GF3, with a degree of polymerization equal to 4; and
a fructo-oligosaccharide containing one glucose unit and four fructose units, also called 1-beta-fructofuranosylnystose or GF4, with a degree of polymerization equal to 5.

25. The cosmetic composition according to claim 24, wherein the at least one native starch is a native corn starch.

26. The cosmetic composition according to claim 25, wherein the at least one native starch is included in an amount ranging from 0.5 to 20% by weight relative to the total weight of the cosmetic composition.

27. The cosmetic composition according to claim 25, wherein the at least one native starch is included in an amount of more than 5% to 20% by weight relative to the total weight of the cosmetic composition.

28. The cosmetic composition according to claim 25, wherein the at least one short-chain fructo-oligosaccharide is included in an amount of at least 0.5% by weight relative to the total weight of the composition.

29. The cosmetic composition according to claim 25, wherein the cosmetic composition is in the form of a dermo-cosmetic product, a hair product, an intimate hygiene product, a dressing and/or a healing product.
